(19) United States
(12) Patent Application Publication
(10) Pub. No.: US 2009/0043337 A1
(43) Pub. Date: Feb. 12, 2009

(54) Suture-Retaining Device And Anchor

(76) Inventor: Daniel L. Martin, Palo Alto, CA (US)

Correspondence Address: PATTERSON & SHERIDAN, L.L.P., 3040 POST OAK BOULEVARD, SUITE 1500, HOUSTON, TX, 77056, US

(21) Appl. No.: 12/187,325

(22) Filed: Aug. 6, 2008

Related U.S. Application Data
(60) Provisional application No. 60/954,558, filed on Aug. 7, 2007.

(51) Int. Cl. A61B 17/04; A61B 19/00

(52) U.S. Cl. 606/232; 606/301; 606/305; 128/898

(57) ABSTRACT

A device for repairing a rotator cuff tendon injury provides a robust means for reattaching the rotator cuff tendon to the underlying humeral head. The device includes a suture-retaining device and an anchor that is mechanically coupled to a cortex of the humerus and the suture-retaining device. The anchor is attached to the cortex of the humerus to provide a robust means of holding the rotator cuff tendon in the desired position against the humeral head. The suture-retaining device, which is positioned in the intramedullary cavity of the humerus, allows a plurality of sutures that are sewn into the rotator cuff tendon to be tied thereto, and allows any suture knots to be located inside the intramedullary cavity.

### CROSS-REFERENCE TO RELATED APPLICATION

This application claims the benefit of U.S. Provisional Application Ser. No. 60/954,558, filed on Aug. 7, 2007.

### BACKGROUND OF THE INVENTION

1. Field of the Invention

Embodiments of the present invention relate generally to surgical repair of the rotator cuff tendon and, more particularly, to devices and methods used in repairing the rotator cuff tendon that has been detached from the humeral head.

2. Description of the Related Art

In surgeries involving repair of a rotator cuff tendon that has been detached from the humeral head, the tendon must be artificially pressed against the humeral head in order to allow the tendon to naturally re-attach to the underlying bone over time. FIGS. 1 and 2 illustrate two prior art techniques for repairing a rotator cuff tendon that has been detached from the humeral head.

FIG. 1 schematically illustrates a rotator cuff tendon 101 secured to a humeral head 102 of a humerus 100 by a plurality of suture anchors 103. Suture anchors 103 are positioned so that sutures 104, which are sewn into rotator cuff tendon 101, position the tendon against region 105 of humeral head 102, where region 105 approximates the original anatomic attachment region of rotator cuff tendon 101, often referred to as the “footprint.” In this way, rotator cuff tendon 101 will grow onto region 105, forming a new bond between rotator cuff tendon 101 and humeral head 102. Issues associated with the use of the technique illustrated in FIG. 1 include anchor displacement, “lift-off” of the rotator cuff tendon, the availability of a limited number of sutures to secure rotator cuff tendon 101 to humeral head 102, and inability to perform this surgery because rotator cuff tendon 101 has retracted too far away from the original anatomic attachment location of rotator cuff tendon 101.

The bone making up humeral head 102 near the normal attachment of the rotator cuff provides a relatively weak base for the insertion of suture anchors 103, since this portion of humerus 100 is relatively porous and soft. Because of this, adequate fixation of suture anchors 103 to the weaker bone material of humeral head 102 is problematic. Namely, anchor displacement from the supporting bone is a common failure mechanism of suture anchors 103, and results from localized fracturing of the surrounding bone material.

Suture anchors 103, when positioned as shown, hold rotator cuff tendon 101 against region 105. However, when the arm containing humerus 100 is raised, “lift-off” of rotator cuff tendon 101 from region 105 may occur, i.e., rotator cuff tendon 101 is temporarily pulled out of contact with region 105. Lift-off is known to inhibit the re-attachment and healing of rotator cuff tendon 101.

The use of suture anchors 103 limits the number of sutures securing rotator cuff tendon 101 to humeral head 102. This is due to the size constraint between suture anchors 103 and the region of humeral head 102 in which the anchors are placed. Also, the placement of many suture anchors 103 is time consuming and tedious for the surgeon. However, fewer sutures provide a less robust connection between rotator cuff tendon 101 and humeral head 102. It is well known that, for a given surgical application, a large number of small diameter sutures are superior to a few large sutures, in terms of holding power in the tissue. In addition, a small number of larger sutures are generally used with suture anchors, leading to larger knot volumes. This is because the knot volume of a suture is roughly proportional to the cube of the suture diameter. Large knots on the surface of the rotator cuff are undesirable as they rub against the overlying acromial bone.

In some situations, bone tunnels may be used to attach sutures to bone and thereby properly position a tendon to be repaired. FIG. 2 schematically illustrates a bone tunnel 203 formed through a humeral head 202 of a humerus 200 after removal of bone material from the humeral head 202. Bone tunnel 203 is formed so that sutures 204, which are sewn into rotator cuff tendon 201, may be threaded through holes formed in a bone structure 206 and thereby position rotator cuff tendon 201 against region 205 for proper healing. A key limitation of this technique is that an already weak bone structure 206 is further weakened as a result of thread holes formed for sutures 204 and is susceptible to breakage before rotator cuff tendon 201 is sufficiently healed.

### SUMMARY OF THE INVENTION

The present invention provides devices and methods used in repairing the rotator cuff tendon. A device according to one embodiment of the invention comprises a suture-retaining device to which sutures sewn into a rotator cuff tendon are to be attached and an anchor that is mechanically coupled to the suture-retaining device. The anchor has a threaded end configured for engagement with a cortex of the humerus to provide firm support for the suture-retaining device.

According to another embodiment of the invention, a device for supporting sutures comprises a head portion for engagement with a plurality of sutures and an adjustable length body portion having a threaded end for engagement with a bone. The adjustable length body portion may comprise a cannulated screw having the threaded end and an internal threaded portion, and a secondary screw engaged with the internal threaded portion and attached to the head portion.

A method according to another embodiment of the invention comprises the steps of anchoring a screw in a cortex of the humerus and attaching a plurality of sutures that have been sewn into the rotator cuff tendon to a suture-retaining device that is mechanically coupled to the screw.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 schematically illustrates a rotator cuff tendon secured to a humeral head of a humerus by a plurality of suture anchors.

FIG. 2 schematically illustrates a rotator cuff tendon secured to a humeral head of a humerus by a bone tunnel.

FIG. 3 depicts a suture-retaining device and anchor for securing a rotator cuff tendon, according to one embodiment of the invention.

FIG. 4 illustrates an anchor and a suture-retaining device used in conjunction with the removal of a region of humeral head, according to an embodiment of the invention.

FIG. 5 is a schematic cross-sectional view of an adjustable length anchor, according to an embodiment of the invention.

FIG. 6A is a schematic view of a suture-retaining device with a through-hole and clamp mechanism, according to an embodiment of the invention.

FIG. 6B is a schematic cross-sectional view of a suture-retaining device with a suture passed through a through-hole.

FIG. 6C illustrates a cross-section of two surfaces of a suture-retaining device, as well as the resultant pressure present throughout a suture compressed between the surfaces.

FIG. 7 is a flow chart summarizing a sequence of steps for securing the rotator cuff tendon to the humeral head, according to embodiments of the invention.

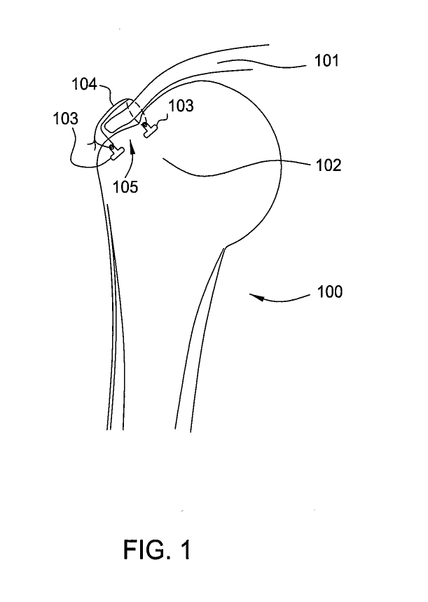

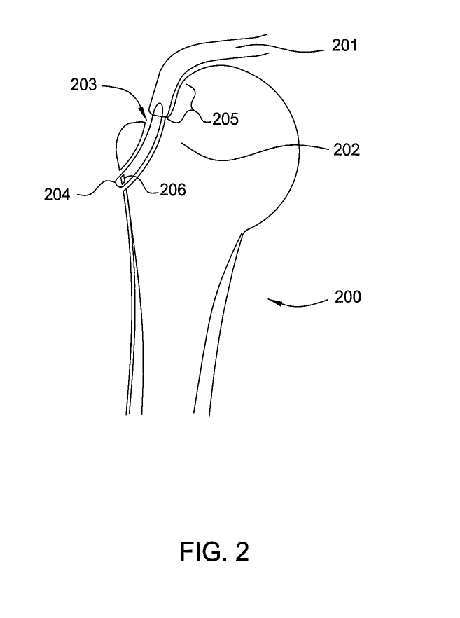

For clarity, identical reference numbers have been used, where applicable, to designate identical elements that are common between figures. It is contemplated that features of one embodiment may be incorporated in other embodiments without further recitation.

### DETAILED DESCRIPTION

Embodiments of the invention contemplate devices and methods used in repairing the rotator cuff tendon that has been detached from the humeral head. A device according to one embodiment of the invention comprises a suture-retaining device and an anchor that is mechanically coupled to the suture-retaining device. The anchor is affixed to a cortex of the humerus to provide firm support for the suture-retaining device. The suture-retaining device, which is positioned in the intramedullary cavity of the humerus, allows a plurality of sutures to connect the rotator cuff tendon to the anchoring device, and allows any suture knots to be located inside the intramedullary cavity.

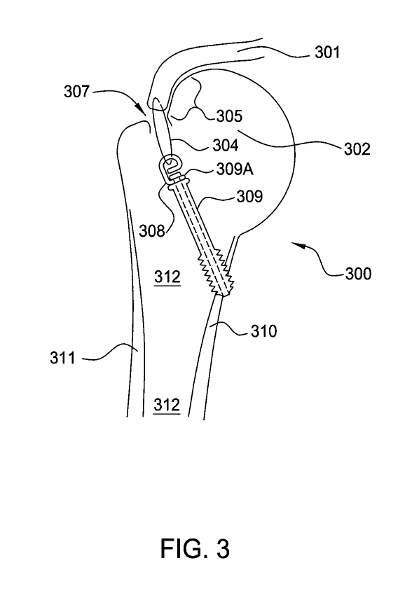

The anchor and suture-retaining device according to embodiments of the invention is positioned in the intramedullary cavity of the humerus after removal of bone material from the humeral head. FIG. 3 depicts a suture-retaining device and anchor for securing the rotator cuff tendon to the humeral head, according to one embodiment of the invention. As shown, a rotator cuff tendon 301 is held against region 305 of humeral head 302. A plurality of sutures 304 are sewn into rotator cuff tendon 301, passed through an opening 307 formed in humeral head 302, looped around a suture-retaining device 308 and tied. For clarity, only one suture 304 is illustrated in FIG. 3, but a large number of sutures is used. Suture-retaining device 308 is a hook or loop configured to accommodate a plurality of sutures, and is mechanically coupled to an anchor 309. In this embodiment, anchor 309 is a bone screw and is threaded into a far cortex 310 of humerus 300. Far cortex 310 is made up of hard bone material and provides a strong base for supporting anchor 309. In this way, anchor displacement due to weak support material is avoided. FIG. 3 depicts anchor 309 inserted into far cortex 310 of humerus 300, but embodiments also contemplate using near cortex 311 as the supporting material for anchor 309.

In the embodiment illustrated in FIG. 3, anchor 309 is a bone screw and suture-retaining device 308 is mechanically coupled to anchor 309 by passing the shaft of anchor 309 through an appropriately sized opening in suture-retaining device 308. In this way, the head 309A of anchor 309 acts as a mechanical stop for suture-retaining device 308. Other means of mechanically coupling suture-retaining device 308 to anchor 309 are also contemplated, including threaded attachment and welding. Suture-retaining device 308 may also be integral with anchor 309 so they are physically inseparable during normal use.

The placement of anchor 309 in far cortex 310 reduces the potential for anchor displacement since far cortex 310 consists of strong bone material. In addition, the positioning of anchor 309 and suture-retaining device 308 in intramedullary cavity 312 of humerus 300, as shown, provides a securing force on rotator cuff tendon 301 at a favorable angle. Because of this, lift-off of rotator cuff tendon 301 from region 305 is minimized, even when the arm containing humerus 300 is raised. Lastly, suture-retaining device 308 facilitates the use of a large number of sutures to secure rotator cuff tendon 301 to anchor 309, therefore smaller sutures with smaller knot volumes may be used and a more robust connection may be established between rotator cuff tendon 301 and anchor 309. For example, a plurality of #0 sutures may be used in place of a single #5 suture.

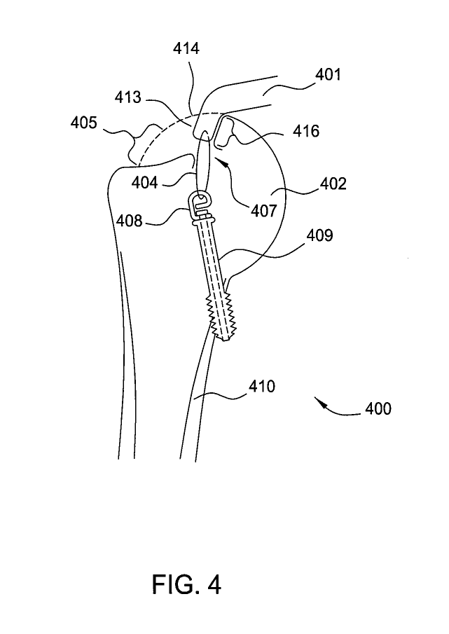

The anchor and suture-retaining device according to embodiments of the invention may be positioned in the intramedullary cavity of the humerus after additional removal of bone material from the humeral head. The additional removal of bone material from the humeral head facilitates repair of a rotator cuff tendon that has been substantially shortened and is unable to reach the footprint of humeral head 302. FIG. 4 illustrates an anchor 409 and a suture-retaining device 408 used in conjunction with the removal of a region 413 of humeral head material, according to an embodiment of the invention. The shape of humeral head 402 prior to removal of region 413 is indicated by original surface 414, which is represented as a broken line. Rotator cuff tendon 401, as shown, is too short for reattachment to region 405 of original surface 414. Therefore, region 413 is removed from humeral head 402, and anchor 409 is inserted into cortex 410 of humerus 400. In this configuration, rotator cuff tendon 401 can be secured against surface 416 of humeral head 402 by sewing a plurality of sutures 404 through rotator cuff tendon 401 and securing the sutures 404 to suture-retaining device 408 via an opening 407 formed in humeral head 402. In this embodiment, suture-retaining device 408 and anchor 409 are substantially similar in organization and function to suture-retaining device 308 and anchor 309, respectively, as described above in FIG. 3. In addition to allowing the repair of a shortened rotator cuff tendon, this embodiment also shares the advantages of the embodiment described above in conjunction with FIG. 3.

The present invention further contemplates a suture-retaining device and an anchor that is configured to have an adjustable length. With a fixed-length anchor, the final position of the suture anchor point relative to the rotator cuff tendon is determined by how far the anchor is inserted into a cortex of the humerus. In some cases, it may be necessary to penetrate the cortex of the humerus with the anchor to the extent that a substantial portion of the anchor protrudes through the cortex. This could be problematic and may result in complications. In contrast, an adjustable length anchor may be placed at an optimal depth in the cortex of the humerus, and the final position of the suture-retaining device may be set by adjusting the length of the anchor. Hence, this embodiment facilitates fine adjustment of the suture-retaining device position relative to a rotator cuff tendon without screwing the anchor too deeply through a cortex of the humerus.

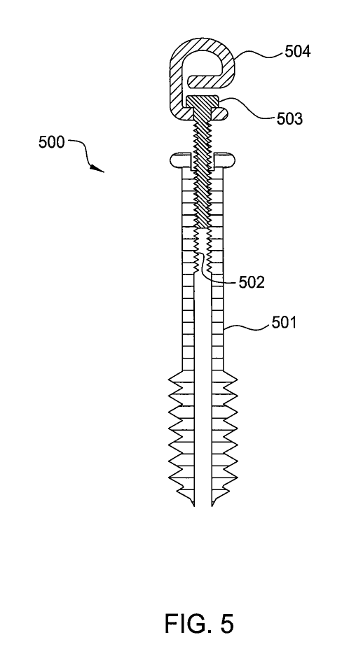

FIG. 5 is a schematic cross-sectional view of an adjustable length anchor 500, according to an embodiment of the invention. Adjustable length anchor 500 includes a cannulated bone screw 501, which has internal threads 502. Internal threads 502 engage with a secondary screw 503, so that secondary screw 503 may adjust the position of a suture-retaining device 504 as desired.

A suture-retaining device may also have alternative configurations according to the present invention. For example, a suture-retaining device may be configured with a clamp mechanism that allows a plurality of sutures to be secured quickly and simultaneously. One time-consuming aspect of tendon repair is the large number of knots that must be tied when securing a tendon to a suture-retaining device. The knots are a limiting factor in the efficiency of the procedure, and with a clamp mechanism, no suture knots are necessary at all. Further, all sutures may be secured at one time.

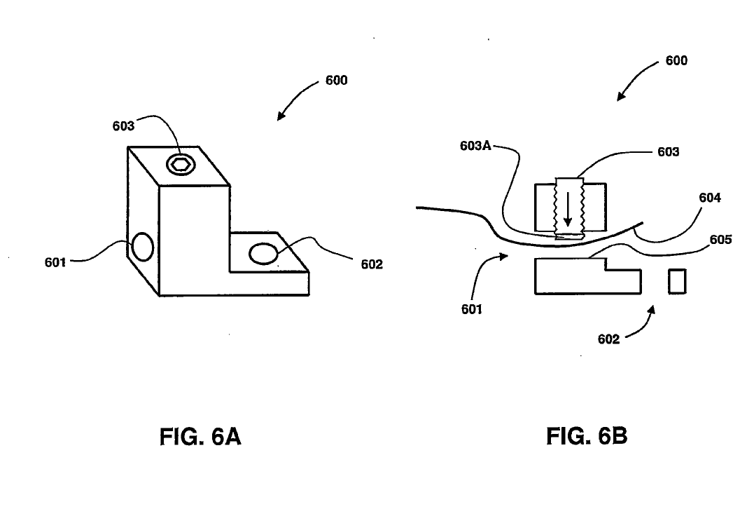

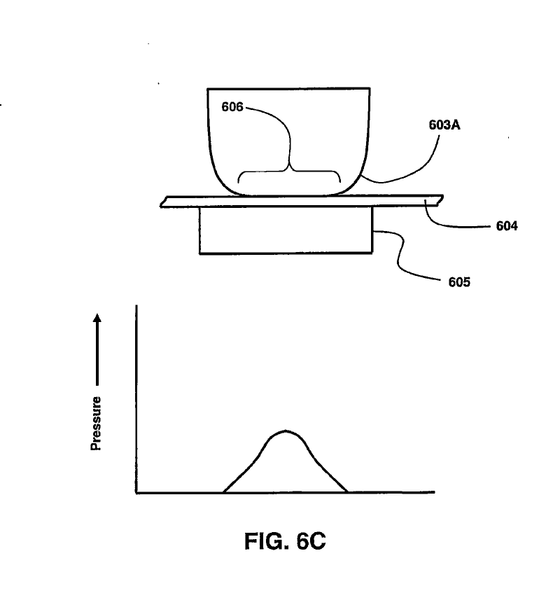

FIG. 6A is a schematic view of a suture-retaining device 600 with a through-hole and clamp mechanism. Suture-retaining device 600 includes a through-hole 601, through which one or more sutures (not shown) is passed. Attachment hole 602 serves as a means for mechanically coupling suture-retaining device 600 to an anchor, such as anchor 309, illustrated in FIG. 3. Hex-head screw 603 may be rotated and moved into through-hole 601 as a clamp to compress sutures positioned therein, obviating the need for suture knots. FIG. 6B is a schematic cross-sectional view of suture-retaining device 600 with a suture 604 passed through through-hole 601. As shown, hex-recess screw 603 may be rotated to move downward against suture 604 and, thus, secure suture 604 to suture-retaining device 600 without the need for a knot. As noted above, a plurality of sutures 604 (not shown for clarity) may be secured by suture-retaining device 600 simultaneously.

To prevent premature separation of suture 604 from suture-retaining device 600, hex-head screw 603 may be configured to minimize cutting of suture 604. In one embodiment, surface 603A of hex-head screw 603 is made up of relatively compliant material, such as a polymer. Alternatively, most or all of hex-head screw 603 may consist of the compliant material. In this embodiment, it is contemplated that the ratio of the modulus of elasticity of the material making up surface 603A to the modulus of elasticity of compression surface 605 is substantially less than about 0.2. In this way, compression of suture 604 between surface 603A and compression surface 605 is less likely to result in cutting of suture 604.

In another embodiment, the cross-section of surface 603A is configured to minimize the pressure gradient experienced by suture 604 when compressed between surface 603A and compression surface 605. A high pressure gradient present on any portion of suture 604, when secured by suture-retaining device 600, might result in cutting of suture 604. Hence, the cross-section of surface 603A is modified to minimize such pressure gradients in suture 604. FIG. 6C illustrates a cross-section of surface 603A and compression surface 605, as well as the resultant pressure present throughout suture 604 when compressed therebetween. As shown, high pressure gradients in suture 604 are additionally avoided by providing surface 603A with a gently tapering profile at the edges of contact region 606. In this embodiment, the profile of surface 603A may be a curve with a gradually changing slope, such as a sinusoidal curve or a section of a circle.

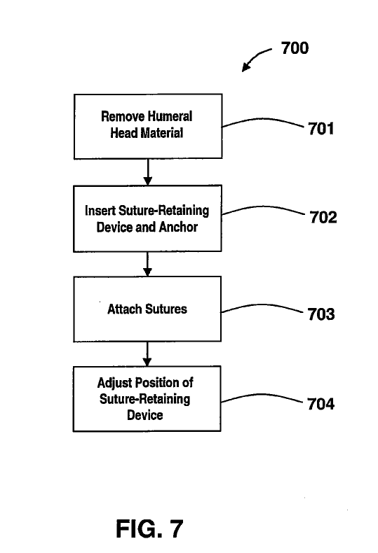

FIG. 7 is a flow chart summarizing a sequence of steps 700 for securing the rotator cuff tendon to the humeral head, according to embodiments of the invention. In step 701, a region of humeral head material is removed to provide access to the intramedullary cavity of the humerus and to facilitate optimal placement of the rotator cuff tendon for reattachment. In step 702, a suture-retaining device and anchor are inserted into the intramedullary cavity of the humerus and the anchor is affixed to a cortex of the humerus. The suture-retaining device is mechanically coupled to the anchor prior to insertion of the anchor. In step 703, sutures are attached to the rotator cuff tendon as necessary and are secured to the suture-retaining device. The sutures may be individually secured to the suture-retaining device by tying. Alternatively, the sutures may all be secured at one time using a through-hole and clamp device as described above in conjunction with FIG. 6. In step 704, the final position of the suture-retaining device may be set by adjusting the depth of penetration of the anchor into the cortex. Alternatively, when the anchor is an adjustable length anchor, adjusting the anchor secondary screw may set the final position of the suture-retaining device.

While the foregoing is directed to embodiments of the present invention, other and further embodiments of the invention may be devised without departing from the basic scope thereof, and the scope thereof is determined by the claims that follow.

What is claimed is:

1. A device used for reattaching a rotator cuff tendon that has detached from a head of a humerus, comprising:
a suture-retaining device to which sutures that are sewn into the rotator cuff tendon are to be attached; and
an anchor that is mechanically coupled to the suture-retaining device and having a threaded end configured for engagement with a cortex of the humerus.

2. The device according to claim 1, wherein the length of the anchor is at least 50 mm.

3. The device according to claim 1, wherein the diameter of the threaded portion is at least 5 mm.

4. The device according to claim 1, wherein the anchor has a head portion to which the suture-retaining device is mechanically coupled.

5. The device according to claim 1, wherein the anchor has an adjustable length.

6. The device according to claim 1, wherein the anchor comprises a cannulated screw with internal threads and a secondary screw that is engaged with the internal threads of the cannulated screw, and wherein the suture-retaining device is mechanically coupled to the secondary screw.

7. The device according to claim 1, wherein the suture-retaining device comprises a loop to which a plurality of sutures can be tied.

8. The device according to claim 1, wherein the suture-retaining device comprises a through-hole in which the sutures are to be received and a clamp that is configured to hold the sutures received in the through-hole in place.

9. The device according to claim 8, wherein the surface of the clamp is polymer.

10. The device according to claim 1, wherein the anchor comprises a bone screw.

11. A device for supporting sutures, comprising:
a head portion for engagement with a plurality of sutures; and
an adjustable length body portion having a threaded end for engagement with a bone.

12. The device according to claim 11, wherein the adjustable length body portion comprises a cannulated screw having the threaded end and an internal threaded portion, and a secondary screw engaged with the internal threaded portion and attached to the head portion.

13. The device according to claim 11, wherein the head portion comprises a loop.

14. The device according to claim 11, wherein the head portion comprises a through-hole into which the sutures are to be inserted and a clamp for holding the sutures inserted into the through-hole in place.

15. A method for repairing a rotator cuff tendon that has detached from a head of a humerus, comprising the steps of:
anchoring a screw in a cortex of the humerus; and
attaching a plurality of sutures that have been sewn into the rotator cuff tendon to a suture-retaining device that is mechanically coupled to the screw.

16. The method according to claim 15, further comprising the steps of forming a hole through the head of the humerus and inserting the screw into the hole prior to the step of anchoring.

17. The method according to claim 16, further comprising the step of mechanically coupling the suture-retaining device to the screw prior to the step of inserting.

18. The method according to claim 15, further comprising the step of adjusting the position of the suture-retaining device relative to the point on the cortex of the humerus at which the screw is anchored.

19. The method according to claim 15, wherein the suture-retaining device is a loop and the sutures are attached to the loop by tying.

20. The method according to claim 15, wherein the suture-retaining device comprises a through-hole and a clamp and the sutures are attached by inserting them into the through-hole and holding them in place with the clamp.
